(19) United States
(12) Patent Application Publication
(10) Pub. No.: US 2007/0293437 A1
(43) Pub. Date: Dec. 20, 2007

(54) Expression Profiling Platform Technology

(76) Inventors: Andras Guttman, San Diego, CA (US); Laszlo Takacs, Newbury Park, CA (US)

Correspondence Address: NIXON & VANDERHYE, PC, 901 NORTH GLEBE ROAD, 11TH FLOOR, ARLINGTON, VA, 22203, US

(21) Appl. No.: 11/665,539

(22) PCT Filed: Oct. 21, 2005
(86) PCT No.: PCT/IB05/03397

§ 371 Date: Apr. 17, 2007

Related U.S. Application Data
(60) Provisional application No. 60/621,423, filed on Oct. 23, 2004.

(51) Int. Cl. A61K 38/00; G01N 33/00; G01N 33/53

(52) U.S. Cl. 514/21; 435/7.21; 435/7.92

(57) ABSTRACT

The present invention relates to an efficient mAb panel-based expression profiling technology platform suitable for global and accurate measurement of proteins, peptides and metabolites in complex mixtures. The platform is comprised of new and well established technologies that are coupled in a unique fashion to provide a novel platform technology for (i) the discovery of differentially displayed elements of complex protein, peptide and metabolite mixtures and (ii) the development of robust mAb based assays that detect the differentially expressed elements.

### INTRODUCTION

The present invention relates to an efficient mAb-based expression profiling technology platform (FIG. 1) suitable for global and accurate measurement of proteins, peptides and metabolites in complex mixtures. The platform is comprised of new and well established technologies that are coupled in a unique fashion to provide a novel platform technology for (i) the discovery of differentially displayed elements of complex protein, peptide and metabolite mixtures and (ii) the development of robust mAb based assays that detect the differentially expressed elements.

Small molecule metabolite, peptide and protein expression analysis is a growing field in the medicinal, veterinarian, food and environmental monitoring and profiling areas. WO2005/077106 relates to a method of identifying biomarkers specific to a disease condition. However, it does not describe the process of specific immunization strategies, analyte library, antibody production, methods of producing monoclonal antibody panels suitable to monitor expression of metabolites and peptides, or the application of these to develop monoclonal antibody arrays. Furthermore, it does not disclose a global approach to the generation of antibody libraries. Precise profiling with the method described herein provides an easy tool for the identification of relevant antigens as well as differences. Moreover, as parallel with the expression analysis and antigen ID, specific mAbs become available; simple or complex antibody based assays are designed for further monitoring and screening only the relevant and differentially expressed elements of the samples. The resulting mAb libraries with their antigen ID could serve as starting panel for the development of simplex or multiplex assays for sensitive, accurate and large scale measurements.

With specific immunization strategies, a large number of mono- or oligoclonal hybridoma supernatants are generated. The entire mAb panel covers the immunogen space of individual protein, peptide or metabolite epitope elements with at least one mAb. Thus, the advanced expression profiling technologies permit the construction of a platform that fulfills three yet unmet needs of protein and metabolite expression analysis: (i) quasi-global coverage, (ii) high level of reproducibility (iii) high sensitivity. Application of cutting edge protein, peptide and metabolite separation technologies for antigen ID completes the technology platform. The high throughput nature and global coverage enable the technology platform to feed into complex data analysis and integration processes.

### SUMMARY OF THE INVENTION

The present invention relates to novel expression profiling methods suitable for global and accurate measurement of proteins, peptides and metabolites in complex mixtures. The invention can be used to generate libraries or panels of monoclonal antibodies specific for blood antigens. It may be used to determine the expression of any analyte from a large variety of complex samples, including samples of human, animal, vegetal or environmental origin. The invention is useful to provide markers, targets, diagnostics and tools useful e.g., in medicinal, veterinarian, food and environmental industries.

A first object of this invention relates to a method of global protein, peptide and/or metabolite expression profiling from a complex analyte sample, comprising of the following steps:
a) Enrichment of the complex analyte sample;
b) Immunization of a non-human mammalian subject with either (i) the enriched complex analyte sample or an aliquot or dilution thereof (shotgun immunization) or (ii) or with a limited analyte library prepared from the enriched complex analyte sample (Limited Immunization); or (iii) with the analyte that is conjugated to a carrier prior to immunization (Conjugated immunization); and
c) Generation of a panel of monoclonal antibodies, derivatives thereof, corresponding hybridomas and/or producing cells, and optionally, analysing the expression profile of said antibodies and/or identifying any antigen bound by antibodies from the panel.

A further aspect of this invention resides in a method for identifying antigens, comprising the steps of:
a) providing at least one complex analyte sample comprising antigens;
b) using said untreated or treated sample as immunogen to immunize a non-human vertebrate, referred to shotgun immunization, preparing monoclonal antibodies, or derivatives thereof, specific for complex analyte sample antigens, from said immunized non-human mammalian, thereby obtaining a panel;
c) profile gene expression at the proteome level by the library or a portion thereof;
d) antigen ID screening of analyte library elements,
e) affinity enrichment of antigens of interest by means of using monoclonal antibodies or derivatives thereof for highly specific recognition;
f) treating affinity enriched sample to fractionate and identify antigens of interest; and
g) identify antigen(s) of interest.

A further aspect of this invention resides in a method of identifying antigens, comprising the steps of:
a) providing at least one complex analyte sample comprising antigens;
b) using the whole or any part of the analyte library, wherein parts share physicochemical or biological properties, as immunogen to immunize a non-human vertebrate (referred to as limited immunization);
c) preparing monoclonal antibodies, or derivatives thereof, specific for complex analyte sample antigens, from said immunized non-human mammalian, thereby obtaining a panel;
d) profile gene expression at the proteome level by the library or a part thereof;
e) antigen ID screening of analyte library elements
f) affinity enrichment of antigens of interest by means of using monoclonal antibodies or derivatives thereof for highly specific recognition;
g) treating affinity enriched sample to fractionate and identify antigens of interest; and
h) identify antigens of interest

A further aspect of this invention resides in a method of monoclonal antibody mediated expression profiling by the generation of many mono or oligoclonal hybridomas, mono or oligoclonal hybridoma supernatants, monoclonal antibodies, a monoclonal antibody panel from nom human vertebrates immunized as discussed above and screening said monoclonal antibody panel for differential reactivity for at least two different samples of complex analytes in a process that involves the following steps. In a particular embodiment, the method further comprises a step of identifying antigens recognized by antibodies or derivatives thereof within said complex sample and/or analyte library, said identification typically comprising the steps below:
Reacting selected monoclonal antibody with complex analyte or element of analyte library, or with a biological sample
Eluting cognate antigen
Identification of cognate antigen via hyphenated chromatography and/or electric field mediated separation process—mass spectrometry or direct peptide sequencing methods.

The complex analyte sample may be any complex sample of biological, environmental, industrial, etc. origin, such as a mixture of proteins and small molecules, e.g.: biological samples like: plasma, serum, urine, body fluids, cell lysates, tissue extracts of human and animal origin. Environmental samples; such as soil, water, cloud condensate, food processing intermediates and food products. Cosmetics and other healthcare products. Any complex mixture that contains immunogen metabolites and/or immunogen proteins, peptides. The complex sample could be a mix of individual complex analyte samples.

In a particular embodiment of the invention, the sample is conjugated to a carrier prior to immunization. In this respect, a further aspect of this invention lies in a method for producing a panel of monoclonal antibodies, comprising the steps of:
a) providing at least one complex analyte sample comprising antigens;
b) partitioning the whole or any part of the analyte library, where parts share physicochemical or biological properties,
c) binding, preferably covalently, library members to a carrier (e.g, a protein) such as, without limitation, albumin, polylysin, keyhole limplet haemocyanin, etc.,
d) using conjugated analyte library member as an immunogen to immunize a non-human vertebrate, referred to as limited immunization; and
e) preparing monoclonal antibodies, or derivatives thereof, specific for complex analyte sample antigens, from said immunized non-human mammalian, thereby obtaining said monoclonal antibody panel.

In a preferred embodiment, the method further comprises the following steps:
f) profile gene expression at the peptidome or metabolome level by the set of monoclonal antibodies, or derivatives thereof;
g) antigen ID screening of analyte library elements
h) affinity enrichment of antigens of interest by means of using monoclonal antibodies or derivatives thereof for highly specific recognition;
i) treating affinity enriched sample to fractionate and identify elements of interest; and identify elements of interest

A further particular object of this invention also resides in a method for producing a library or panel of monoclonal antibodies, or derivatives thereof, specific for human blood antigens, the method comprising the steps of:
a) providing at least one biological sample comprising human blood antigens;
b) treating said sample under conditions allowing depletion of abundant proteins while retaining low abundant proteins present in normal human blood;
c) using said treated sample or a portion thereof as an immunogen to immunize a non-human mammalian; and
d) preparing monoclonal antibodies, or derivatives thereof, specific for human blood antigens, from said immunized non-human mammalian, thereby obtaining said mAb panel.

The invention also relates to a mAb panel obtainable by such a method, as well as to any uses thereof.

### LEGEND TO THE FIGURES

FIG. 1: Shotgun immunization based protein, peptide and metabolite expression profiling platform

FIG. 2: SDS PAGE electrophoresis of treated human plasma analyte, 5 ug of complex protein mix was loaded to each well, a 4-20% acrylamide gel was run and stained with silver nitrate.

FIG. 3: Monoclonal antibodies are captured in the ELISA assay by a goat anti Ig-Fc antibody. Captured antibodies are incubated with biotinylated plasma samples. Reaction is developed by peroxidase coupled avidin complexes. Peroxidase reaction is visualized via OPD and the reaction is measured at 492 nm in an ELISA reader. To detect individual proteins a direct ELISA reaction was applied on pure target proteins and on the eluate of the Aglient column.

FIG. 4: Comassie blue stained SDS PAGE gel. Numbers in parenthesis show the sequence coverage obtained by MALSI-TOF MS/MS.

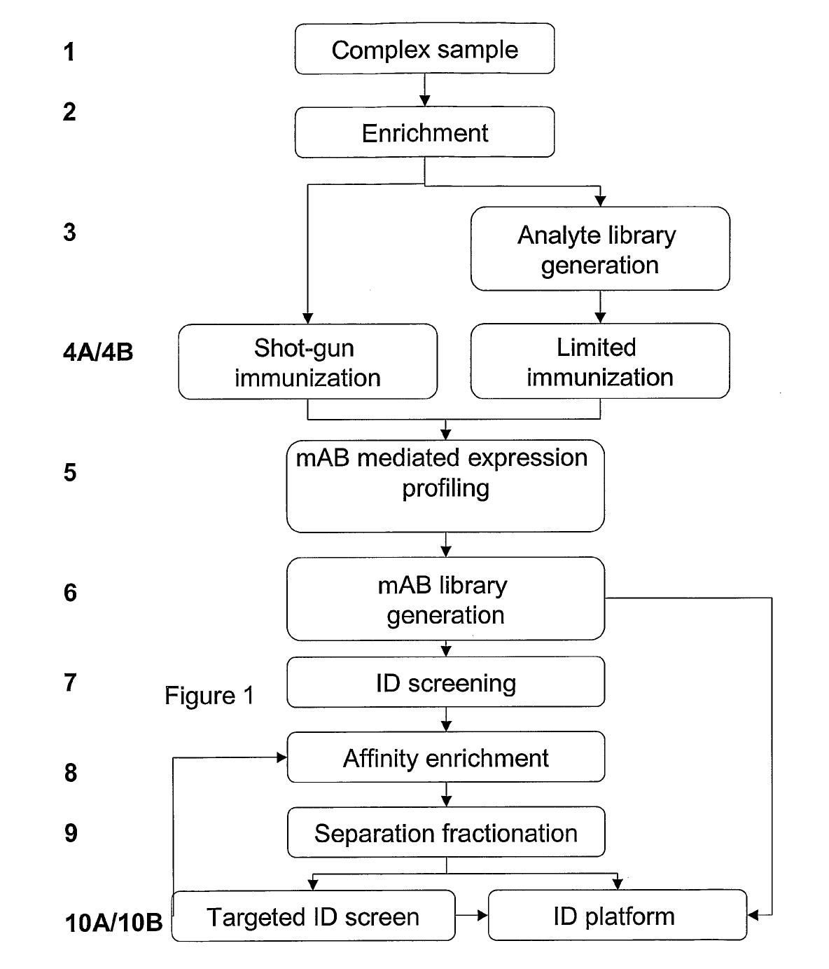

### DETAILED DESCRIPTION OF THE INVENTION

The Platform:

As discussed above, a first object of this invention relates to a method of global protein peptide and/or metabolite expression profiling from a complex analyte sample, comprising of the following steps:
a) Enrichment of the complex analyte sample;
b) Immunization of a non-human mammalian subject with either (i) the enriched complex analyte sample or an aliquot or dilution thereof (shotgun immunization) or (ii) or with a limited analyte library prepared from the enriched complex analyte sample (Limited Immunization); or (iii) with the analyte conjugated to a carrier prior to immunization (Conjugated immunization) and
c) Generation of a panel of monoclonal antibodies, derivatives thereof, corresponding hybridomas and/or producing cells, and optionally, analysing the expression profile of said antibodies and/or identifying any antigen bound by antibodies from the panel.

The non human vertebrate may be any non human mammal, such as but not limited to a rodent, a rabbit, or a chicken.

A further particular object of this invention is a method of monoclonal antibody mediated expression profiling by the generation of many mono or oligoclonal hybridomas, mono or oligoclonal hybridoma supernatants, monoclonal antibodies, a monoclonal antibody panel from nom human vertebrates immunized as disclosed above and screening said monoclonal antibody panel for differential reactivity for at least two different samples of complex analytes. The process preferably involves the following steps:
Generation of a panel of monoclonal antibodies or derivatives thereof, wherein said panel comprises a plurality of containers comprising annotated monoclonal antibodies, corresponding to monoclonal or oligoclonal antibody producing hybridomas, specific for distinct analyte elements, e.g. human blood antigens. Moreover, wherein said panel comprises antibodies, or derivatives thereof, those e.g., which bind to low abundant antigens from diseased and from healthy human subjects.

The complex analyte preferably comprises of at least two clinical samples. The clinical samples may represent at least two disease conditions or at least one drug-responding group and one non-responding group or disease susceptible and non-susceptible individuals or diseased and apparently healthy subjects such as but not limited to cancer patients before and after tumor resection or cancer patients with and without recurrence of primary cancer, or cancer patients with and without metastasis.

According to specific embodiments, the clinical sample is selected from human serum or plasma, human urine, human sputum, human brochoalveolar fluid, human biopsy material, human tissue section, human faeces and human exudates.

In a specific embodiment, the monoclonal antibody panel is screened via ELISA assay.

In a further specific embodiment, the monoclonal antibody panel is immobilized to a surface, such as a solid surface, in particular but not limited to glass, silicon, plastic, membrane (and is screened e.g., as microarray). Alternatively, the monoclonal antibody panel may be immobilized to any gel for screening. The monoclonal antibody panel may be screened in multiplex antibody arrays comprising fluorescent antibody conjugated beads. In a particular embodiment, the monoclonal antibody panel is screened via chemiluminescent assay.

The density of the array may be variable, and adjusted by the skilled artisan. Typical densities include a density ranging from 10-1,000/cm² to about 1,000-1,000,000/cm².

1. Complex Samples:

Mixture of proteins and small molecules, e.g., but not limited to: biological samples like: plasma, serum, urine, body fluids, cell lysates, tissue extracts of human and animal origin. Environmental samples; such as soil, water, cloud condensate, food processing intermediates and food products. Cosmetics and other healthcare products. Any complex mixture that contains immunogen metabolites and/or immunogen proteins, peptides. The complex sample could be a mix of individual complex analyte samples.

In order to enrich for differentially expressed elements of two or multiple complex samples to be compared; the samples will be enriched for the those elements that are of special interest (see below)

2. Enrichment by Partitioning

Some elements of complex samples are irrelevant for subsequent analysis, either because these do not carry information relative to the question that drives the analysis, and/or their abundance is so high that it interferes and jeopardizes the analysis process (e.g. albumin in human plasma samples). Affinity chromatography will be used to eliminate these elements. The partitioning process may be mediated by individual monoclonal antibodies, mixtures of monoclonal antibodies, polyclonal antiserum or ligands to which undesired elements will bind. Either the depleted fraction or the eluate of affinity chromatography process might be used for further steps.

A specific example: Immunosorbent chromatography, e.g.: columns from GenWay, Inc or Agilent Inc. are used to remove the most abundant serum and plasma proteins. Alternatively, anti-human serum could be used to remove elements of the complex human mixture that are both relatively highly abundant, and present or enriched only in healthy individuals. Moreover, polyclonal antisera prepared to specific mixtures or proteins or metabolites, metabolite classes could be applied either to enrich or to deplete these from the complex analyte mixture.

In a specific embodiment, the enrichment is carried out by treating the sample to partition. Preferably, the treating comprises separation technology; affinity enrichment e.g., using antibodies; organic ligand affinity chromatography; ion exchange chromatography; hydrophobic interaction chromatography; hydrophilic interaction chromatography; electrophoresis; size exclusion chromatography; chromatofocusing; isoelectrofocusing or a combination thereof.

3. Analyte Library Generation

(Multidimensional Separations Based on Physical, Chemical and Biochemical Characteristics, Differential Display)

In order to support downstream analyte identification (ID) processes, complex mixtures are separated into specific classes via a suitable multidimensional and hierarchic separation process that is based on physical, chemical and biochemical characteristics of the complex mixture. The result of this step is a hierarchic set of pools; within the pool of complex mixtures the individual elements share at least one common characteristics. Process proximal pools differ in complexity from process distal pools and share fewer characteristics. Process ultimate pools might contain only a single type/class of element that is apparently homogeneous and contains no or only trace amount of less related contaminating elements. These ultimate pools if loaded to identification process (e.g. mass spectrometry based protein ID) will provide a single ID or a very likely one.

Screening of pools with mab-s (ID screen) provides information on the shared physical, chemical and biochemical characteristics.

Analyte library is not necessarily the same as the one that is being profiled.

Differential display analysis of labeled samples from the separated fractions: Comparison of analyte libraries allows the identification of apparent differences at the level of pools in complexity levels and relative representation of individual elements. These pools being differentially displayed, could be applied preferentially for the immunization process (Limited immunization).

For limited immunization, an analyte library is generated, typically by separating analytes having common physicochemical or biological properties. Typically, all elements recovered and placed to individual containers.

4. 4.A. Shot-Gun Immunization

Immunization with complex protein sample: Either the enriched fraction or the complex protein mixture is used to immunize mice. Immunization is done by the use of well established technologies.

4.B. Limited Immunization

Immunization with analyte pools. To increase the chances of obtaining monoclonal hybridomas reactive with all immunogen elements, lower complexity analyte pools are used for immunization. These pools could contain proteins, peptides or metabolites that share at least one important physical chemical or biochemical characteristics. As described in 3A, proteins are used for immunization directly, while peptides, and metabolites are used after derivatization to adjuvant immunogens.

In a specific embodiment of limited immunization, non human mammals are immunized with a one or more elements of the analyte library.

In particular embodiments, more than two elements of the analyte library share at least one physicochemical characteristic and/or at least one biochemical characteristic and/or at least one immunochemical property and/or at least one affinity binding capacity and/or are homologous in their peptide sequence.

4.C. Conjugated Immunization

Immunization with complex peptide mixtures or complex metabolites, or individual peptide or metabolites: This step involves derivatization of an adjuvant immunogen carrier protein or artificial adjuvant immunogenic polymer in fashion that permits conjugation of peptides or metabolites. Metabolites and peptides are coupled to the adjuvant immunogen via their reactive groups in separate conjugation processes, e.g.: one process for OH esters, another for NH2 groups etc. Finally derivatized adjuvant immunogens are mixed and the mixture is used to immunize mice

5. Monoclonal Antibody (mAb) Mediated Expression Profiling of Individual Samples

High sensitivity micro-ELISA assays are designed that use the monoclonal hybridoma supernatant and labeled complex tracers derived from the complex sample or its pools. Thousands of mAb containing hybridoma supernatants are tested in a screen to identify those that discriminate individual classes of analyte samples (e.g. derived from disease vs. healthy individuals, or treated vs. non treated groups, etc.).

6. Monoclonal Antibody Panel Generation

After rigorous statistical calculations a panel of mAb containing hybrodomas are selected. Large scale mAb generation could be initiated for each selected hybridoma at this step. The panel is subjected to downstream steps in order to identify “ID” each immunogen antigen that reacts with an individual mAb in panel.

The antibody derivatives may be an antibody fragment, preferably selected from Fab, Fab′, CDR, and single chain antibodies (ScFv). The antibody derivative is preferably a human or humanized antibody or fragment thereof. Such derivatives may be produced by any method known per se in the art.

One way to produce humanized antibodies is to isolate the cDNA of a particular mouse monoclonal antibody, sequence the cDNA region coding for the peptide region that binds to the antigen. One can directly sequence this region via peptides sequencing technologies. In the next step the region is cloned into the similar region of a human antibody cDNA and expressed. Particular care should be paid to engineer the regions of glycosilation to ensure that these are human like. The step above can be applied to the heavy chain or to the light chain or to both. An alternative way is to produce monoclonal antibodies from transgenic mice that carry a part of or the entire human antibody gene sets, but may not have mouse antibody genes. These mice produce human antibodies and may not produce mouse antibodies

7. ID Screen

This step (optional) involves the screening of analyte library pools in a hierarchic and economic manner to identify the pool that contains the antigen recognized by the monoclonal antibody, or the monoclonal antibody containing hybridoma supernatant. If necessary, affinity enrichment and targeted screening steps are deployed to identify the antigen.

8. Affinity Enrichment

Affinity enrichment that can be but not limited to column or microbead based processes. The relevant mAb's generated during steps 1-5 and screened positive in Step 6 are immobilized to appropriate stationary phase material or micro-bead substrate and used as bait for antigen purification. This process can be repeated as many times as necessary to collect the required amount of material for downstream processing.

9. Separation and Fractionation

The separation and fractionation of the enriched eluent from Step 8 can be accomplished but not limited to liquid chromatography (LC), capillary electrophoresis (CE), capillary electrochromatography (CEC), microchip based analytical methods or other separation technologies. The collected fractions or split eluent stream is being interrogated in Step 10 for activity in a mAb mediated screen (targeted screening)

10. Targeted Screening
(i) (10A) One part of the split flow is used for screening the fractions of Step 8 for antibody reactivity with the appropriate mAb, and collected for ID analysis in step 10B.
(ii) (Loop to Step 8) Break the mAb/AG complex and reprocess the mAb in Step 8.
(iii)(10B) Mass spectrometry (MS and MSⁿ) based identification using but not limited to ESI or MALDI ionization methods. In case of proteins, digestion, separation and MSⁿ. In case of metabolites, separation and MSⁿ.

In a particular embodiment, the method of ID screening comprises a step of identifying antigens recognized by antibodies or derivatives thereof within said complex sample and/or analyte library where identification comprises the steps below:
Reacting selected monoclonal antibody with complex analyte or element of analyte library, or with a biological sample
Eluting cognate antigen
Identification of cognate antigen via hyphenated chromatography and/or electric field mediated separation system—mass spectrometry or direct peptide sequencing methods.

The identification of said antigens typically comprise contacting an antibody or derivative thereof with a biological sample and determining the identity of an antigen specifically bound to said antibody or derivative thereof. Identification of the cognate antigen may be preceded by screening the entire or part of the analyte library in order to identify the source of material for the cognate antigen ID. Alternatively, or in addition, identification of the cognate antigen may be preceded by affinity enrichment of the antigen and/or by partitioning, including but not limited to separation and fractionation.

In a particular embodiment, the process is an automated platform, and/or involves one or more microfluidic or micro total analysis system (μTAS) chip

Production of a Panel (or Library) of Antibodies Specific for Human Blood Antigens

A particular object of this invention resides in a method for producing a library or panel of monoclonal antibodies, or derivatives thereof, specific for human blood antigens, the method comprising the steps of:
a) providing at least one biological sample comprising human blood antigens;
b) treating said sample under conditions allowing depletion of abundant proteins while retaining low abundant proteins present in normal human blood;
c) using said treated sample or a portion thereof as an immunogen to immunize a non-human mammalian; and
d) preparing monoclonal antibodies, or derivatives thereof, specific for human blood antigens, from said immunized non-human mammalian, thereby obtaining said panel (or library).

According to a preferred embodiment, the method further comprises a step of profiling antibodies, or derivatives thereof, within the panel against one or several control or diseased samples, to obtain an annotated panel of monoclonal antibodies, or derivatives thereof. The profiling step typically comprises determining whether the antibody or derivative thereof specifically binds an antigen contained in a blood sample from a control or diseased human subject.

In a particular embodiment, the profiling step comprises determining whether the antibody or derivative thereof binds an antigen present in at least one control sample and two diseased samples.

The method of this invention preferably further comprises a step of identifying antigens recognized by antibodies or derivatives thereof within said panel. The identification of said antigens typically comprises contacting an antibody or derivative thereof from the panel with a biological sample and determining the identity of an antigen specifically bound to said antibody or derivative thereof.

As discussed above, the antibody derivative is e.g., an antibody fragment, preferably selected from Fab, Fab′, CDR and single chain antibodies (ScFv). The antibody derivative is preferably a human or humanized antibody or a fragment thereof.

The biological sample comprising human blood antigens typically is or derives from a human plasma sample, a human serum sample or a human blood sample. The biological sample is preferably derived from a healthy human subject.

In a particular embodiment of the method, the panel comprises monoclonal antibodies or derivatives thereof produced from biological samples from different human subjects. The biological samples may all derive from several healthy subjects, or from several healthy and diseased subjects.

In step b) above, the sample is typically contacted with an affinity column that removes from 2 to 22 most abundant human proteins. The depleted sample preferably comprises between about 5 to 10% of total human serum proteins.

In step c) above, the whole treated sample, in aliquots, may be used as an immunogen, or different classes of antigens present in the sample are separated, and separate immunizations are performed with each of said classes.

The panel can comprise a plurality of monoclonal antibodies, producing hybridomas and/or derivatives thereof, which are arranged in separate containers. In this respect, a particular object of this invention also resides in a panel (or library) of monoclonal antibodies, or derivatives thereof, wherein said panel comprises a plurality of containers comprising annotated monoclonal antibodies, or derivatives thereof, or corresponding producing hybridomas, specific for distinct human blood antigens, wherein said panel comprises antibodies, or derivatives thereof, that bind low abundant antigens from diseased and from healthy human subjects.

The invention also concerns a product comprising, immobilized on a support, preferably in an ordered manner, a plurality of monoclonal antibodies, or derivatives thereof, specific for distinct human blood antigens, wherein said product comprises antibodies, or derivatives thereof, that bind low abundant antigens from diseased and from healthy human subjects. As discussed above, the support may be a solid or semi-solid material, such as a membrane, glass, plastic, ceramic or metal support having a surface, or a gel.

Such a panel of mAbsor product may be used to identify markers, therapeutic antibodies, to design diagnostic kits, etc. In this respect, a particular object of this invention also concerns a method for identifying antibodies that bind a selected target, the method comprising contacting said target with all or a portion of a panel of monoclonal antibodies, or derivatives thereof, as defined above or obtainable by a method as defined above, or with a product as defined above, under conditions allowing an antigen-antibody reaction to occur, and identifying one or several monoclonal antibodies, or derivatives thereof, from said mAb panel, that bind said target.

The invention also relates to a method for identifying antibodies that are specific for a particular condition or disease, the method comprising contacting a biological sample from a mammalian having said condition or disease with all or a portion of a panel of monoclonal antibodies, or derivatives thereof, as defined above or obtainable by a method as defined above, or with a product as defined above, under conditions allowing an antigen-antibody reaction to occur, and identifying one or several monoclonal antibodies, or derivatives thereof, from said library, that form antibody-antigen complexes with said sample.

The invention further relates to a method for identifying one or several mammalian antigens specific to a condition or disease, the method comprising contacting a biological sample from a mammalian having said condition or disease with all or a portion of a panel of monoclonal antibodies, or derivatives thereof, as defined above or obtainable by a method as defined above, or with a product as defined above, under conditions allowing an antigen-antibody reaction to occur, identifying one or several monoclonal antibodies, or derivatives thereof, from said panel, that form antibody-antigen complexes with said sample and, identifying antigens engaged into said complexes.

Further aspects and advantages of the present invention will be disclosed in the following examples, which should be considered as illustrative and not limiting the scope of this application.

### EXAMPLES

### I—Enrichment by Partitioning

A complex analyte sample (human plasma) is treated to partition. Highly abundant proteins are separated here from medium and low abundant ones via affinity chromatography using a commercial chromatography system with a multiaffinity removal column (Agilent). Highly abundant proteins are those that are represent in the plasma at a concentration level that is higher than 1 mg/ml. The experiment is performed as described in the Agilent Technologies manual. In the example, the enriched sample contains minimal, trace, or not detectable concentrations of the following proteins: human serum albumin, IgA, haptoglobin, anti-trypsin, IgG, transferrin

To test the efficiency of enrichment, specific protein analytes were coated onto plastic plates and specific mAbs were used to detect the bound analyte species (e.g. Apolipoprotien A1, complement factor C3, IgA). The amount of specific mAb bound is in direct correlation with the analytes species quantity bound to the plastic surface, mAb binding was visualized by horse radish peroxidase coupled rabbit anti-mouse Ig. Standard curves were used to calculate relative abundance level, which is expressed in fractions (%) of total protein content of the analyte. Results show that only traces of IgA is detectable, while other analytes species are enriched.

TABLE 1
Relative abundance level of various protein analyte
species before and after the treatment.

| | Before | After |
|---|---|---|
| | Depletion (%) | depletion (%) |
| ApoA1 | 1.1 | 16.5 |
| C3 | 0.5 | 20 |
| IgA | 1.25 | 0.32 |

### II—Analyte Library Generation

Analyte libraries are generated from complex analyte mixtures that may contain proteins, peptides and/or metabolites at the same time. Multidimensional separation technology is applied to partition all elements or specific classes of elements, e.g. proteins from all metabolites. Moreover, complex separation processes can also be applied e.g. those that retain the proteins in their antigenic conformations, yet allow separation of classes or individual types of proteins. Separation technologies that use caotripic agents e.g, detergents, e.g. SDS are usually irreversibly denaturing, prevent the analyte to react with antibodies that exclusively recognize the natural conformation. High concentration of organic solvents and the process of binding and elution from separation surfaces are denaturing as well. Thus a carefully selected process is applied that conserves antigenic determinants. An initial step could be affinity chromatography with polyclonal antibody directed against components of the complex analyte.

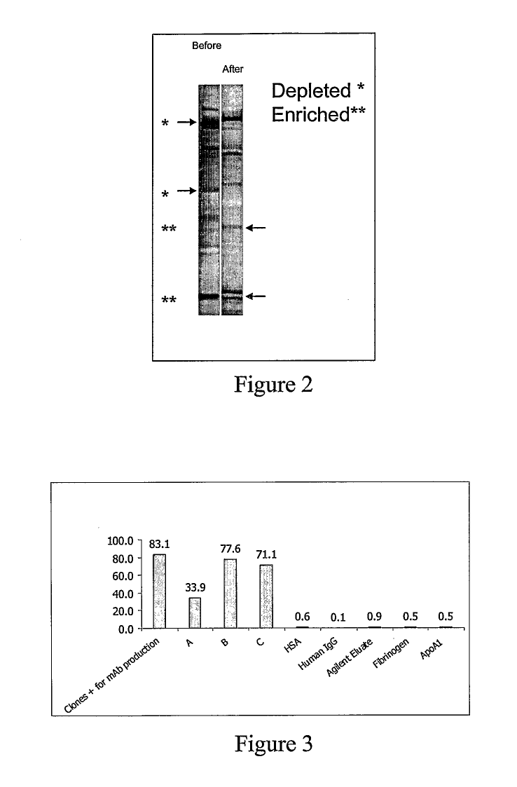

To determine the efficiency and progress of analyte library preparation, SDS PAGE electrophoresis separation was done on samples that have undergone affinity chromatography separation mediated by a polyclonal antibody directed to the human serum. The separation step clearly enriches some elements while some others are virtually eliminated. (FIG. 2).

### III—Shotgun Immunization, mAb Mediated Expression Profiling

Complex enriched or enriched and treated analyte mix directly (protein samples) or after conjugation en-mass to immunoadjuvant carriers (peptide and metabolite samples) are injected into mice or other species that develops a clonable antibody repertoire. In the example, treated complex analyte mix, human plasma, was injected into Balbc female mice in the presence of complete Freund adjuvant first, then bi-weekly in the presence of incomplete adjuvant. Injections were done into the footpad and s.c. at multiple places. The last injection was done i.v. without adjuvant. Three days after the last injection spleen cells were fused to Sp2Ag0 mouse hybridoma partner cells in the presence of 50% Polyethylene glycol. Fused cells were cultured in 96 well plates in the presence of selection medium. One thousand one hundred eighty five hybridoma supernatants from this antibody panel were screened in ELISA assays. As shown on FIG. 3. 83.1% of the supernatants produced antibodies. Three different plasma preparations have been tested. It is evident, that 77.6 percent of the clones react with plasma proteins in sample A, only 33.9 in sample B, while 71.1% react with sample C, 14.5% of clones positive with B, give higher signal with C than B while 75.1% of clones positive with sample A give higher signal with B than A. The majority of the mAB panel elements thus detect representational differences in protein level and together this is suitable for protein expression profiling. Utility of a plasma proteome specific mAb panel or library is dependent on the level of redundancy, e.g. the number of antibody clones directed against the most abundant proteins in the plasma. We have tested some of those that are removed from the complex analyte by the analyte treatment method described in example-I. Results are shown for serum albumin, IgG, fibrinogen and apolipoprotein A1 and the eluate of the Agilent depletion column (FIG. 3). Clones specific for these abundant proteins are represented at <1% frequency.

### IV—Affinity Enrichment Separation Fractionation, ID Screening

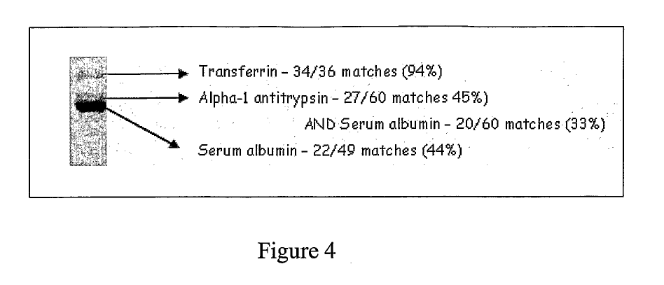

One possible regimen for protein ID is affinity purification followed by SDS gel electrophoresis and mass spectrometry (MS) on material derived from gel slices containing a single stainable band. In FIG. 4 we show that apparent purity (single band one gel slice) is sufficient criteria for good quality protein ID. In the complex analyte sample we applied onto SDS PAGE electrophoresis multiple proteins were present, still bands cut out from the gel provided single proteins and high quality ID, as analyzed by MALDI-TOF MS technology. As described below: Gel bands were rinsed with an ammonium bicarbonate buffer/acetonitrile 1:1 mixture in order to eliminate the gel stain (CBB) and SDS. The proteins were reduced by DTT, the free sulfhydrils were derivatized with iodoacetamide. Then the proteins were incubated with side chain protected porcine trypsin (Promega) for 4 hrs at 37° C. The resulting peptides were extracted and zip-tip purified. The unfractionated digests were subjected to MALDI-TOF MS analysis using 2,5-dihydroxy-benzoic acid (DHB) as matrix. An MS-Fit http://prospector.ucsf.edu) database search was performed with the masses detected against the NCBI nonredundant protein database NCBInr.2005.01.06. During the search no species restriction was applied. The identity of the proteins was verified by post source decay (PSD) analysis of a selected peptide. The protein MWs and pIs obtained represented the full length sequences as listed in the database-that do not necessarily reflect the size of the expressed, especially the processed, mature, and active proteins.

1-93. (canceled)

94. A method of global protein peptide and/or metabolite expression profiling of a complex analyte sample, comprising the following steps:
a) Enrichment of the complex analyte sample;
b) Immunization of a non-human mammalian subject with either (i) the enriched complex analyte sample or an aliquot or dilution thereof (shotgun immunization) or (ii) with a limited analyte library prepared from the enriched complex analyte sample (Limited Immunization) or (iii) with the analyte that is conjugated to a carrier prior to immunization (Conjugated immunization); and
c) Generation of a panel of monoclonal antibodies, derivatives thereof, corresponding hybridomas and/or producing cells, and optionally, analysing the expression profile of said antibodies and/or identifying any antigen bound by antibodies from the mAb panel.

95. A method of claim 94, wherein in the enrichment step the complex analyte sample is treated to partition.

96. A method of claim 95, wherein said treatment to partition comprises a separation technology or affinity enrichment, preferably using antibodies.

97. A method of claim 95, wherein said treatment treating comprises organic ligand affinity chromatography, ion exchange chromatography, hydrophobic interaction chromatography, hydrophilic interaction chromatography, electrophoresis, size exclusion chromatography, Chromatofocusing, or isoelectrofocusing, or a combination thereof.

98. A method for shotgun immunization based antigen identification, comprising the steps of:
a) providing at least one complex analyte sample comprising antigens;
b) using said untreated or treated sample as immunogen to immunize a non-human vertebrate, referred to shotgun immunization, preparing monoclonal antibodies, or derivatives thereof, specific for complex analyte sample antigens, from said immunized non-human mammalian, thereby obtaining a library of antibodies;
c) profiling gene expression at the proteome level by the library of antibodies, or derivatives thereof;
d) antigen ID screening of analyte library elements
e) affinity enrichment of antigens of interest by means of using monoclonal antibodies or derivatives thereof for highly specific recognition;
f) treating affinity enriched sample to fractionate and identify elements of interest; and
g) identifying elements of interest.

99. A method of claim 98, wherein the complex analyte is treated by partitioning and all elements are recovered and placed to individual containers.

100. A method of claim 99, wherein treating comprises a separation technology or affinity enrichment, preferably using antibodies, organic ligand affinity chromatography, ion exchange chromatography, hydrophobic interaction chromatography, hydrophilic interaction chromatography, electrophoresis, size exclusion chromatography, Chromatofocusing, or isoelectrofocusing, or a combination thereof.

101. A method of identifying antigens, comprising the steps of:
a) providing at least one complex analyte sample comprising antigens;
b) using the complex analyte sample or an analyte library comprising parts of the analyte sample, where parts share physicochemical or biological properties, as immunogen to immunize a non-human vertebrate; and
c) preparing monoclonal antibodies, or derivatives thereof, specific for complex analyte sample antigens, from said immunized non-human mammalian, thereby obtaining a library of monoclonal antibodies;
d) optionally profiling gene expression at the proteome level by the set of monoclonal antibodies, or derivatives thereof;
e) antigen ID screening of analyte library elements;
f) affinity enrichment of antigens of interest by means of monoclonal antibodies or derivatives thereof for highly specific recognition;
g) treating affinity enriched sample to fractionate; and
h) identifying antigens of interest.

102. A method of claim 101, wherein non human vertebrates are immunized with a treated or non treated complex analyte.

103. A method of claim 94, wherein the non human vertebrate is a non human mammal, preferably a rodent, a rabbit or a chicken.

104. A method of claim 101, wherein non human mammals are immunized with one or more elements of the analyte library.

105. A method of claim 101, wherein more than two elements of the analyte library share at least one physicochemical, biochemical and/or immunochemical characteristic, and/or affinity binding capacity, and/or are homologous in their peptide sequence.

106. A method of monoclonal antibody mediated expression profiling, comprising the generation of monoclonal hybridomas, mono or oligoclonal hybridoma supernatants, monoclonal antibodies or a monoclonal antibody panel from non human vertebrates immunized as in claim 94, and screening said monoclonal antibody panel for differential reactivity for at least two different samples of complex analytes.

107. A method for identifying antibodies that bind to or differentially bind to an analyte sample, the method comprising contacting said analyte sample with all or a portion of a library of monoclonal antibodies, or derivatives thereof, as defined or obtainable by a method of claim 94, under conditions allowing an antigen-antibody reaction to occur, and identifying one or several monoclonal antibodies, or derivatives thereof, from said library, that bind said analyte sample.

108. A method for identifying antibodies that are specific for a particular condition or disease, the method comprising contacting a biological sample from a mammalian having said condition or disease with all or a portion of a library of monoclonal antibodies, or derivatives thereof, as defined or obtainable by a method of claim 94, under conditions allowing an antigen-antibody reaction to occur, and identifying one or several monoclonal antibodies, or derivatives thereof, from said library, that form antibody-antigen complexes with said sample.

109. A method of claim 94, wherein the complex analyte comprises of at least two clinical samples.

110. A method of claim 109, wherein said clinical samples represent at least two disease conditions, at least two groups of drug responding and non-responding individuals, disease susceptible and non-susceptible individuals, diseased and apparently healthy subjects, cancer patients before and after primary tumor or tumor metastasis resection, cancer patients with and without recurrence of primary cancer, or cancer patients with and without metastasis.

111. A method of claim 94, wherein said clinical sample is human serum or plasma, or human urine, sputum, bronchoalveolar fluid, biopsy material, tissue section, faeces or exudates.

112. A method of claim 94, wherein said monoclonal antibody panel is screened via ELISA assay.

113. A method of claim 95, wherein said monoclonal antibody panel is immobilized to a solid surface, preferably a glass surface, silicon surface or plastic surface, and screened as microarray, or wherein said monoclonal antibody panel is immobilized to any gel, or membrane, such as lipid membranes, for screening.

114. A method of one of claim 94, wherein said monoclonal antibody panel is screened in multiplex antibody arrays comprising fluorescent antibody conjugated beads, or via chemiluminescent assay, or via assays that do not use label.

115. A method of claim 113, wherein the density of the array is between 10-1,000/cm², or between 1,000-1,000,000/cm².

116. A method of claim 106, wherein the profiling step comprises determining whether the antibody or derivative thereof binds an antigen present in at least one analyte sample.

117. The method of claim 94, wherein the ID screening comprises a step of identifying antigens recognized by antibodies or derivatives thereof within said complex sample and/or analyte library, wherein identification comprises the steps below:
Reacting selected monoclonal antibody with complex analyte or element of analyte library, or with a biological sample,
Eluting cognate antigen, and
Identification of cognate antigen via hyphenated chromatography and/or electric field mediated separation methods—mass spectrometry or direct peptide sequencing methods.

118. The method of claim 117, wherein the identification of said antigens comprises contacting an antibody or derivative thereof with a biological sample and determining the identity of an antigen specifically bound to said antibody or derivative thereof.

119. The method of claim 94, wherein the complex analyte sample comprises a mixture of proteins and small molecules, and is preferably selected from a biological sample, such as plasma, serum, urine, body fluids, cell lysates, tissue extracts of human and animal origin; an environmental sample, such as soil, water, cloud condensate; food processing intermediates and food products; cosmetics and other healthcare products; any complex mixture that contains immunogen metabolites and/or immunogen proteins or peptides; or a combination thereof.

120. A method of claim 117, wherein identification of the cognate antigen is preceded by screening the entire or part of the analyte library in order to identify the source of material for the cognate antigen ID or by affinity enrichment of the antigen, or by partitioning, including but not limited to separation and fractionation.

121. A method of claim 94, wherein the process is an automated platform.

122. A method of claim 94, wherein the process involves one or more microfluidic chip or micro total analysis system (μTAS).

123. A method of providing a library of antibodies, comprising the steps of:
a) providing at least one complex analyte sample comprising antigens;
b) partitioning the whole or any part of the analyte library, where parts share physicochemical or biological properties,
c) conjugating the analyte to a carrier prior to immunization, such as albumin, polylysin or keyhole limplet haemocyanin,
d) using the conjugated analyte library member as immunogen to immunize a non-human vertebrate; and
e) preparing monoclonal antibodies, or derivatives thereof, specific for complex analyte sample antigens, from said immunized non-human mammalian, thereby obtaining said panel.

124. A method of claim 123, whereby the complex analyte is treated by partitioning and all elements are recovered and placed to individual containers.

125. A method of claim 124, wherein treating comprises a separation technology or affinity enrichment, preferably using antibodies, organic ligand affinity chromatography, ion exchange chromatography, hydrophobic interaction chromatography, hydrophilic interaction chromatography, electrophoresis, size exclusion chromatography, Chromatofocusing, or isoelectrofocusing, or a combination thereof.

126. A method for producing a panel of monoclonal antibodies, or derivatives thereof, specific for human blood antigens, the method comprising the steps of:
a) providing at least one biological sample comprising human blood antigens;
b) treating said sample to deplete abundant proteins while retaining low abundant proteins present in normal human blood;
c) using said treated sample or a portion thereof as an immunogen to immunize a non-human mammalian; and
d) preparing monoclonal antibodies, or derivatives thereof, specific for human blood antigens, from said immunized non-human mammalian, thereby obtaining said panel.

127. The method of claim 126, further comprising:
a step of profiling antibodies, or derivatives thereof, within the library against one or several control or diseased samples, to obtain an annotated panel of monoclonal antibodies, or derivatives thereof; and/or
a step of identifying antigens recognized by antibodies or derivatives thereof within said mAb panel.

128. The method of claim 127, wherein the profiling step comprises determining whether the antibody or derivative thereof specifically binds an antigen contained in a blood sample from a control or diseased human subject, and/or determining whether the antibody or derivative thereof binds an antigen present in at least one control sample and two diseased samples.

129. The method of claim 126, wherein the identification of said antigens comprise contacting an antibody or derivative thereof from the mAb panel with a biological sample and determining the identity of an antigen specifically bound to said antibody or derivative thereof.

130. The method of claim 94, wherein the antibody derivative is an antibody fragment, preferably selected from Fab, Fab′, CDR and single chain antibodies (ScFv), further preferably is a human or humanized antibody or fragment thereof.

131. The method of claim 126, wherein the biological sample comprising human blood antigens is or derives from a human plasma sample, a human serum sample or a human blood sample.

132. The method of claim 126, wherein the biological sample is derived from a healthy human subject.

133. The method of claim 126, wherein the panel comprises monoclonal antibodies or derivatives thereof produced from biological samples from different human subjects, typically from several healthy subjects or from several healthy and diseased subjects.

134. The method of claim 126, wherein, in step b), the sample is contacted with an affinity column that removes from 1 to 50, preferably 2 to 22 most abundant human proteins.

135. The method of claim 126, wherein the depleted sample comprises between about 0.02 to 25% of total human serum proteins, more preferably less than 5%.

136. The method of claim 126, wherein the whole treated sample, in aliquots, is used as an immunogen.

137. The method of claim 126, wherein different classes of antigens present in the sample are separated, and separate immunizations are performed with each of said classes.

138. The method of claim 126, wherein the panel comprises a plurality of monoclonal antibodies, producing hybridomas and/or derivatives thereof, which are arranged in separate containers.

139. A library or panel of monoclonal antibodies, or derivatives thereof, wherein said panel comprises a plurality of containers comprising annotated monoclonal antibodies, or derivatives thereof, or corresponding producing hybridomas, specific for distinct human blood antigens, wherein said panel comprises antibodies, or derivatives thereof, that bind low abundant antigens from diseased and from healthy human subjects.

140. A method for identifying antibodies that bind a selected target, the method comprising contacting said target with all or a portion of a panel of monoclonal antibodies, or derivatives thereof, as defined in claim 139 or obtainable by a method of:
a) providing at least one biological sample comprising human blood antigens;
b) treating said sample to deplete abundant proteins while retaining low abundant proteins present in normal human blood;
c) using said treated sample or a portion thereof as an immunogen to immunize a non-human mammalian; and
d) preparing monoclonal antibodies, or derivatives thereof, specific for human blood antigens, from said immunized non-human mammalian, thereby obtaining said panel, under conditions allowing an antigen-antibody reaction to occur, and identifying one or several monoclonal antibodies, or derivatives thereof, from said panel, that bind said target.

141. A method for identifying antibodies that are specific for a particular condition or disease, the method comprising contacting a biological sample from a mammalian having said condition or disease with all or a portion of a panel of monoclonal antibodies, or derivatives thereof, as defined in claim 139 or obtainable by a method of:
a) providing at least one biological sample comprising human blood antigens;
b) treating said sample to deplete abundant proteins while retaining low abundant proteins present in normal human blood;
c) using said treated sample or a portion thereof as an immunogen to immunize a non-human mammalian; and
d) preparing monoclonal antibodies, or derivatives thereof, specific for human blood antigens, from said immunized non-human mammalian, thereby obtaining said panel, under conditions allowing an antigen-antibody reaction to occur, and identifying one or several monoclonal antibodies, or derivatives thereof, from said panel, that form antibody-antigen complexes with said sample.

142. A method for identifying one or several mammalian antigens specific to a condition or disease, the method comprising contacting a biological sample from a mammalian having said condition or disease with all or a portion of a panel of monoclonal antibodies, or derivatives thereof, as defined in claim 139 or obtainable by a method of:
a) providing at least one biological sample comprising human blood antigens;
b) treating said sample to deplete abundant proteins while retaining low abundant proteins present in normal human blood;
c) using said treated sample or a portion thereof as an immunogen to immunize a non-human mammalian; and
d) preparing monoclonal antibodies, or derivatives thereof, specific for human blood antigens, from said immunized non-human mammalian, thereby obtaining said panel, under conditions allowing an antigen-antibody reaction to occur, identifying one or several monoclonal antibodies, or derivatives thereof, from said panel, that form antibody-antigen complexes with said sample and, identifying antigens engaged into said complexes.
